SEQUENCE LISTING (33 sequences)

SEQ ID NO: 1 - protein, 16 aa, Artificial Sequence
  Synthetic Construct
KSSQSLLYSRGKTYLN

SEQ ID NO: 2 - protein, 7 aa, Artificial Sequence
  Synthetic Construct
AVSKLDS

SEQ ID NO: 3 - protein, 9 aa, Artificial Sequence
  Synthetic Construct
VQGTHYPFT

SEQ ID NO: 4 - protein, 10 aa, Artificial Sequence
  Synthetic Construct
GYTFTRYYIN

SEQ ID NO: 5 - protein, 16 aa, Artificial Sequence
  Synthetic Construct
INPGSGNTKYNEKFKG

SEQ ID NO: 6 - protein, 6 aa, Artificial Sequence
  Synthetic Construct
EGTTVY

SEQ ID NO: 7 - protein, 10 aa, Artificial Sequence
  Synthetic Construct
GYTFTDYYIN

SEQ ID NO: 8 - protein, 6 aa, Artificial Sequence
  Synthetic Construct
EGETVY

SEQ ID NO: 9 - protein, 11 aa, Artificial Sequence
  Synthetic Construct
RASKSISKYTA

SEQ ID NO: 10 - protein, 7 aa, Artificial Sequence
  Synthetic Construct
AGSKRHW

SEQ ID NO: 11 - protein, 9 aa, Artificial Sequence
  Synthetic Construct
QQHNEYPYT

SEQ ID NO: 12 - protein, 10 aa, Artificial Sequence
  Synthetic Construct
GYAFTSFLIE

SEQ ID NO: 13 - protein, 16 aa, Artificial Sequence
  Synthetic Construct
SNPRTGRTKYKSKFRG

SEQ ID NO: 14 - protein, 5 aa, Artificial Sequence
  Synthetic Construct
EFFDY

SEQ ID NO: 15 - protein, 7 aa, Artificial Sequence
  Synthetic Construct
AGSKLHW

SEQ ID NO: 16 - protein, 16 aa, Artificial Sequence
  Synthetic Construct
SNPRTGGRKYKEKFRG

SEQ ID NO: 17 - protein, 16 aa, Artificial Sequence
  Synthetic Construct
RSSQSLLISGGKTYLN

SEQ ID NO: 18 - protein, 7 aa, Artificial Sequence
  Synthetic Construct
LVSKLDQ

SEQ ID NO: 19 - protein, 9 aa, Artificial Sequence
  Synthetic Construct
WQGTYFPLT

SEQ ID NO: 20 - protein, 10 aa, Artificial Sequence
  Synthetic Construct
GKTFWSYGIN

SEQ ID NO: 21 - protein, 16 aa, Artificial Sequence
  Synthetic Construct
IYIGTGYTEPNPKYKG

SEQ ID NO: 22 - protein, 10 aa, Artificial Sequence
  Synthetic Construct
IGGYYGNFDQ

SEQ ID NO: 23 - protein, 11 aa, Artificial Sequence
  Synthetic Construct
KASDHIGKFLT

SEQ ID NO: 24 - protein, 7 aa, Artificial Sequence
  Synthetic Construct
GATSKLT

SEQ ID NO: 25 - protein, 9 aa, Artificial Sequence
  Synthetic Construct
QQYWSTPFT

SEQ ID NO: 26 - protein, 10 aa, Artificial Sequence
  Synthetic Construct
GYKFTRYVMH

SEQ ID NO: 27 - protein, 16 aa, Artificial Sequence
  Synthetic Construct
INPYNDGVNYNEKFKG

SEQ ID NO: 28 - protein, 6 aa, Artificial Sequence
  Synthetic Construct
NWDTGL

SEQ ID NO: 29 - protein, 11 aa, Artificial Sequence
  Synthetic Construct
KASDHILKFLT

SEQ ID NO: 30 - protein, 7 aa, Artificial Sequence
  Synthetic Construct
GATSLET

SEQ ID NO: 31 - protein, 9 aa, Artificial Sequence
  Synthetic Construct
QMYWSTPFT

SEQ ID NO: 32 - protein, 10 aa, Artificial Sequence
  Synthetic Construct
GYKFTRYVMH

SEQ ID NO: 33 - protein, 16 aa, Artificial Sequence
  Synthetic Construct
INPYNDGTNYNEKFKG

(19) United States
(12) Patent Application Publication
(10) Pub. No.: US 2023/0236199 A1
(43) Pub. Date: Jul. 27, 2023

(54) SUBCUTANEOUS ABSORPTION AND BIOAVAILABILITY OF ANTIBODIES

(71) Applicant: Eli Lilly and Company, Indianapolis, IN (US)

(72) Inventors: Selina Alyssa Lynette Estwick, Greenwood, IN (US); Amita Datta-Mannan, Indianapolis, IN (US)

(21) Appl. No.: 17/905,580

(22) PCT Filed: Apr. 16, 2021
(86) PCT No.: PCT/US2021/027767

§ 371 Date: Sep. 2, 2022

Related U.S. Application Data
(60) Provisional application No. 63/014,225, filed on Apr. 23, 2020.

(51) Int. Cl. G01N 33/68; A61K 9/00

(52) U.S. Cl. CPC G01N 33/6845; A61K 9/0019

(57) ABSTRACT

Provided herein are methods of selecting antibodies suitable for subcutaneous administration; methods of improving subcutaneous absorption and bioavailability of antibodies; and methods of administering an antibody to a subject subcutaneously.

### FIELD OF THE INVENTION

The present disclosure relates to methods of selecting antibodies suitable for subcutaneous administration; methods of improving subcutaneous absorption and bioavailability of antibodies; and methods of administering an antibody to a subject subcutaneously.

### BACKGROUND

Over the last few decades, human or humanized monoclonal antibody (mAb) pharmaceuticals have been successfully used as therapeutic modalities in a wide array of human diseases due to their target binding specificity, bivalent interaction properties, potential to have innate effector function and their in vitro and in vivo biochemical stability (Kaplon H and Reichert J M. mAbs 2018; 10:183-203; Kaplon et al., MAbs. 2020 Jan.-Dec.; 12(1):1703531). Advances in antibody engineering methods such as humanization, potency and specificity optimization for achieving the ideal pharmacodynamics (PD) and improvements in the drug-ability properties, such as pharmacokinetics (PK), are vital to the success of mAb based therapies.

Relative to intravenous (IV) route, SC administration is generally preferred for therapeutic antibodies in clinical settings due to increased patient convenience and compliance (Matucci A, et al., Respir Res 2018; 19:154; Viola M, et al., J Control Release 2018; 286:301-14). Often times, a barrier to this endeavor includes bioavailability limitations associated with SC injection that reduce systemic exposure. The bioavailability of mAbs has been difficult to predict following SC administration, can be variable and partial with values of ˜50 to 100% (Lobo E D, et al., J Pharm Sci 2004; 93:2645-68; Turner M R and Balu-Iyer S V. J Pharm Sci 2018; 107:1247-60; Wang W, et al., Clin Pharmacol Ther 2008; 84:548-58). Currently, the average SC bioavailability for marketed mAbs in humans is ˜60-80% (Viola M, et al., J Control Release 2018; 286:301-14; Turner M R and Balu-Iyer S V. J Pharm Sci 2018; 107:1247-60; Richter W F and Jacobsen B. Drug metabolism and disposition: the biological fate of chemicals 2014; 42:1881-9). While the mechanisms related to the incomplete bioavailability observed for some mAbs are not well understood, there is a general consensus that the PK fate and absorption profile of mAbs following SC administration requires an understanding of the SC space/anatomy and composition.

The SC matrix or hypodermis has been reviewed extensively (Viola M, et al., J Control Release 2018; 286:301-14; Turner M R and Balu-Iyer S V. J Pharm Sci 2018; 107:1247-60; Richter W F and Jacobsen B. Drug metabolism and disposition: the biological fate of chemicals 2014; 42:1881-9). After SC administration the mAb has to be shunted through the interstitium to reach these capillaries. Given the mixture of cells (adipocytes, macrophages and fibroblasts) and matrices (adipose, glycosaminoglycans (GAGs), proteoglycans, elastin and collagen), it is possible that the PK fate, absorption profile and engineering strategies for improving mAb SC kinetics requires an understanding of the interplay of the molecule's physiochemical properties with the SC space and anatomy.

Interestingly, while the number of mAb based biological therapies have increased, there is still quite a bit of debate and a paucity of information around the relative balance between physiochemical characteristics and their impact on mAb PK. This has led to an inadequate understanding on how these parameters might affect absorption processes for mAbs administered to the SC space. Some limited physiochemical elements such as the molecular weight and FcRn binding capacity have been interrogated. In addition, some studies have been conducted with charge based mAb variants with mixed findings. In a study by Khawli et. al., no significant differences in SC absorption were reported with IgG1 charge variants; however, the pI of these IgG1 molecules varied marginally (within 0.1 pI units) in this report and thereby may not have been different enough to affect SC absorption (Khawli L A, et al., mAbs 2010; 2:613-24). In contrast, in another report, mAbs with a broader range of pI (1 unit differences), showed a moderate trend correlating increasing mAb pI and decreasing SC bioavailability (Zheng Y, et al., mAbs 2012; 4:243-55). Consistent with the later findings, Mach and coworkers reported that positively charged mAbs interact in vitro with SC tissue likely mediated via electrostatic interactions (Mach H, et al., Ther Deliv 2011; 2:727-36). Taken together, these handful of studies lay some foundation on the role of intrinsic mAb physiochemical features on the rate and extent of SC absorption. There is a scarcity of data for factors such as the hydrophobicity, thermal stability and aggregation potential of mAbs following SC injection, as well as, the interplay of these factors with charge, isoelectric point and FcRn binding interactions.

There exists a need for selecting mAbs suitable for subcutaneous administration and improving subcutaneous absorption and bioavailability of mAbs.

### DETAILED DESCRIPTION

Provided herein are methods of selecting antibodies (e.g., mAbs) suitable for subcutaneous administration; methods for improving subcutaneous absorption and bioavailability of antibodies (e.g., mAbs); and methods of administering an antibody (e.g., mAb) to a subject subcutaneously.

In one aspect, provided herein are methods of selecting an antibody (e.g., mAb) suitable for subcutaneous administration, such methods comprise measuring T_{agg} (temperature of aggregation onset) of a first and a second antibody that binds to the same target, measuring T_{m onset} (temperature of the unfolding onset) of the first and second antibody, comparing the T_{agg} and T_{m onset} of the first and second antibody; and selecting the first or second antibody that has a higher T_{agg} and/or T_{m onset} for subcutaneous administration. In some embodiments, such methods further comprise measuring HpnIP (heparin binding interaction potential) and/or HIP (hydrophobic interaction potential) of the first and second antibody. In some embodiments, such methods further comprise selecting the first or second antibody that has a lower HpnIP and/or HIP. In some embodiments, such methods further comprise measuring the rate of subcutaneous absorption (ka) and/or subcutaneous bioavailability (% F) of the first and second antibody.

In some embodiments, such methods further comprise measuring one or more of the PK parameters of the first and second antibody, wherein the PK parameters are selected from Cₘₐₓ (maximal observed serum concentration), Tₘₐₓ (time of maximal observed serum concentration), AUC_{0-inf} (area under the serum concentration curve from time zero extrapolated to infinite time), CL/F (clearance following SC administration), and T_{1/2} (elimination half-life).

In some embodiments, the first antibody and the second antibody are both monoclonal antibodies, e.g., humanized mAbs. In some embodiments, the first antibody and the second antibody have an IgG1 or IgG4 isotype. In some embodiments, the first antibody and the second antibody both comprise a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3.

In some embodiments, such methods further comprise evaluating if the first and second antibody comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid; the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia. Based on the observations in the Examples, inclusion of one or more of the specified amino acids in a particular CDR suggests the antibody may have a favorable subcutaneous absorption and bioavailability profile.

In some embodiments, such methods comprise selecting the first or second antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid; the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

In some embodiments, such methods further comprise evaluating if the first and second antibody comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

In some embodiments, such methods further comprise selecting the first or second antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

Provided herein are also antibodies selected for subcutaneous administration by any of the methods described herein. In some embodiments, the antibody is a monoclonal antibody, e.g., a humanized mAb. In some embodiments, the antibody has an IgG1 or IgG4 isotype.

In another aspect, provided herein are methods of generating a variant antibody with improved subcutaneous absorption and bioavailability compared to a parental antibody (e.g., mAb), such methods comprise generating a variant antibody of the parental antibody, wherein the variant antibody has a higher T_{agg} and/or T_{m onset} than the parental antibody. In some embodiments, wherein the variant antibody has a lower HpnIP and/or HIP than the parental antibody. In some embodiments, such methods further comprise measuring ka and/or % F of the parental antibody and the variant antibody. In some embodiments, such methods further comprise measuring one or more of the PK parameters of the parental antibody and the variant antibody, wherein the PK parameters are selected from Cₘₐₓ, Tₘₐₓ, AUC_{0-inf}, CL/F, and T_{1/2}.

In some embodiments, the parental antibody and the variant antibody are both monoclonal antibodies, e.g., humanized mAbs. In some embodiments, the parental antibody and the variant antibody have an IgG1 or IgG4 isotype. In some embodiments, the parental antibody and the variant antibody both comprise a heavy chain variable region (VH) and a light chain variable region (VL), wherein the VH comprises heavy chain complementarity determining regions (HCDR) HCDR1, HCDR2, and HCDR3, and the VL comprises light chain complementarity determining regions (LCDR) LCDR1, LCDR2, and LCDR3.

In some embodiments, such methods further comprise generating a variant antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid;
the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

In some embodiments, the methods of generating a variant antibody further comprise:
replacing the amino acid residue at position 24 of LCDR1 of the parental antibody with lysine;
replacing the amino acid residue at position 54 of LCDR2 of the parental antibody with leucine;
replacing the amino acid residue at position 55 of LCDR2 of the parental antibody with aspartic acid or glutamic acid;
replacing the amino acid residue at position 56 of LCDR2 of the parental antibody with serine or threonine;
replacing the amino acid residue at position 96 of LCDR3 of the parental antibody with phenylalanine; or
replacing the amino acid residue at position 61 of HCDR2 of the parental antibody with glutamic acid;
wherein all positions are numbered according to Kabat numbering and the CDRs are defined by a hybrid of Kabat and Chothia.

In some embodiments, such methods further comprise generating a variant antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

In some embodiments, the methods of generating a variant antibody further comprise:
replacing the amino acid residue at position 25 of LCDR1 of the parental antibody with alanine or serine;
replacing the amino acid residue at position 26 of LCDR1 of the parental antibody with serine;
replacing the amino acid residue at position 52 of LCDR2 of the parental antibody with serine or threonine;
replacing the amino acid residue at position 89 of LCDR3 of the parental antibody with glutamine or valine;
replacing the amino acid residue at position 90 of LCDR3 of the parental antibody with glutamine;
replacing the amino acid residue at position 95 of LCDR3 of the parental antibody with proline;
replacing the amino acid residue at position 97 of LCDR3 of the parental antibody with threonine;
replacing the amino acid residue at position 26 of HCDR1 of the parental antibody with glycine;
replacing the amino acid residue at position 27 of HCDR1 of the parental antibody with tyrosine;
replacing the amino acid residue at position 29 of HCDR1 of the parental antibody with phenylalanine;
replacing the amino acid residue at position 30 of HCDR1 of the parental antibody with threonine;
replacing the amino acid residue at position 62 of HCDR2 of the parental antibody with lysine; or
replacing the amino acid residue at position 65 of HCDR2 of the parental antibody with glycine;
wherein all positions are numbered according to Kabat numbering and the CDRs are defined by a hybrid of Kabat and Chothia.

Provided herein are also variant antibodies generated by any of the methods described above. In some embodiments, the variant antibody is a monoclonal antibody, e.g., a humanized mAb. In some embodiments, the variant antibody has an IgG1 or IgG4 isotype.

In another aspect, provided herein are methods of selecting an antibody suitable for subcutaneous administration, the method comprising selecting an antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid; the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

In some embodiments, such methods further comprise selecting an antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

In some embodiments, such methods further comprise measuring T_{agg} and T_{m onset} of the antibody. In some embodiments, such methods further comprise measuring HpnIP and/or HIP of the antibody. In some embodiments, such methods further comprise measuring ka and/or % F of the antibody. In some embodiments, such methods further comprise measuring one or more of the PK parameters of the antibody, wherein the PK parameters are selected from Cₘₐₓ, Tₘₐₓ, AUC_{0-inf}, CL/F, and T_{1/2}.

In a further aspect, provided herein are methods of administering an antibody (e.g., mAb, e.g., humanized mAb) to a subject subcutaneously; such methods comprise: measuring T_{agg} and T_{m onset} of the antibody, determining the antibody is suitable for subcutaneous administration, and subcutaneously administering the antibody to the subject. In some embodiments, such methods further comprise measuring HpnIP and/or HIP of the antibody. In some embodiments, such methods further comprise measuring ka and/or % F of the antibody. In some embodiments, such methods further comprise measuring one or more of the PK parameters of the antibody, wherein the PK parameters are selected from Cₘₐₓ, Tₘₐₓ, AUC_{0-inf}, CL/F, and T_{1/2}.

As used herein, the term “a,” “an,” “the” and similar terms used in the context of the present disclosure (especially in the context of the claims) are to be construed to cover both the singular and plural unless otherwise indicated herein or clearly contradicted by the context.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). The CDR definition used herein is a hybrid of Kabat and Chothia.

Exemplary embodiments of antibodies of the present disclosure also include antibody fragments or antigen-binding fragments, which comprise at least a portion of an antibody retaining the ability to specifically interact with an antigen such as Fab, Fab′, F(ab′)2, Fv fragments, scFv, scFab, disulfide-linked Fvs (sdFv), a Fd fragment and linear antibodies.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

The term “subject”, as used herein, refers to a mammal, including, but are not limited to, a human, chimpanzee, ape, monkey, cattle, horse, sheep, goat, swine, rabbit, dog, cat, rat, mouse, guinea pig, and the like. Preferably the subject is a human.

The term “therapeutically effective amount,” as used herein, refers to an amount of a protein or nucleic acid or vector or composition that will elicit the biological or medical response of a subject, for example, reduction or inhibition of an enzyme or a protein activity, or ameliorate symptoms, alleviate conditions, slow or delay disease progression, or prevent a disease, etc. In a non-limiting embodiment, the term “a therapeutically effective amount” refers to the amount of a protein or nucleic acid or vector or composition that, when administered to a subject, is effective to at least partially alleviate, inhibit, prevent and/or ameliorate a condition, or a disorder or a disease.

As used herein, “treatment” or “treating” refers to all processes wherein there may be a slowing, controlling, delaying or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### Examples

### Materials and Methods

Construction, Expression and Purification of the Six mAbs

The Fab regions were cloned into mAb expression vectors to fuse with constant regions of human kappa light chain and either human IgG₁ or IgG₄ heavy chain using standard molecular biology approaches and confirmed by DNA sequencing. All the IgGs were expressed using a CHO expression system. The mAbs were purified from culture supernatants using standard Protein-A Sepharose (GE Healthcare) affinity chromatography followed by size exclusion chromatography methods described previously (Datta-Mannan A, et al., mAbs 2015; 7:483-93).

### Evaluation of the Cynomolgus Monkey FcRn Binding Affinity

Recombinant soluble cynomolgus monkey (cFcRn) was expressed in 293EBNA cells transfected with plasmids encoding for the soluble portion of αFcRn and β₂-microglobulin, and the protein was purified as described previously (Datta-Mannan A, et al., Drug metabolism and disposition: the biological fate of chemicals 2007; 35:86-94; Datta-Mannan A, et al., The Journal of biological chemistry 2007; 282:1709-17). The interaction of the IgG₁ and IgG₄ molecules with recombinant, immobilized cFcRn were monitored by SPR detection using a Biacore 3000 instrument (GE Healthcare) as described previously (Datta-Mannan A, et al., Drug metabolism and disposition: the biological fate of chemicals 2007; 35:86-94; Datta-Mannan A, et al., The Journal of biological chemistry 2007; 282:1709-17). Briefly, recombinant soluble cFcRn was immobilized to flow cell 2 of a CM5 sensor chip using amine coupling chemistry (GE Healthcare). The cFcRn immobilization surface density was approximately 300 RU. The first flow cell was used as a blank control surface lacking cFcRn. All binding experiments were performed with compounds dissolved in running buffer phosphate buffered saline (PBS) with 0.005% Tween 20, pH 6 or PBS with 0.005% Tween 20, pH 7.4 and the samples were run at a flow rate of 100 μl/min for 30 seconds with a dissociation time of 10 minutes. PBS (pH 7.4) was used as dissociation buffer. PBS with 0.005% Tween 20, pH 6 was used as running buffer for the experiments performed to determine the affinity of IgGs to cFcRn. A concentration range of 0.00316 μM to 3.16 μM of each of the IgGs was used to estimate the association and dissociation constants. The binding data were obtained by subtracting the signal of flow cell 1 (blank flow cell not coupled with FcRn) from flow cell 2. Kinetic (association and dissociation) data were then simultaneously fit to a heterogeneous binding model for IgG-cFcRn interactions (BIAevaluation, Ver. 4.1). The data curves for binding and dissociation phases of the sensorgrams for the IgGs at pH 6.0 had low residuals and low x values. The mean of K_{d} values accounting for the greatest fraction of binding from two independent experiments were reported.

Evaluation of mAb Isoelectric Points (pIs)

Capillary Isoelectric Focusing (cIEF) method was used to measure pI of all mAbs. All protein samples were diluted to 1 mg/mL with 10 mM citrate at pH 6. The final protein concentration was diluted to 0.25 mg/mL by the cIEF master solution which includes 4% pH 3-10 pharmalyte and 4 M urea. Maurice*(Protein Simple, San Jose, Calif.) was used for data acquisition and analysis, which were achieved through the compass for ice software (Version: 1.1.5 Build ID: 0920). During the data acquisition, the markers of 4.1 and 9.5 were used and separation of various charged species were done by applying 1500 volts for 1 minute followed by 3000 volts for 9 minutes. After acquisition, the raw data were processed by correct the marker position. The peak with the highest intensity and area within the chromatogram was assigned as the pI value of the protein.

Zeta potentials of 1 mg/mL mAb solutions in either 10 mM histidine pH6 or 10 mM acetate pH5 were measured by electrophoretic light scattering with a Zetasizer® (Malvern Instruments Ltd., UK) instrument. At 25° C., the particle refractive index was set at 1.003. Solution refractive index, viscosity, and dielectric constant were calculated based on the solution components using Zetasizer software.

### Evaluation of Temperature of Melting or Midpoint of Temperature Transition (Tₘ) and the Onset Temperature of Tertiary Structure Unfolding

A MicroCal VP-Capillary differential scanning calorimetry (DSC) system (Malvern Instruments Ltd., Malvern UK) was used for midpoint of temperature transition (Tₘ) measurement. Samples were diluted to 1 mg/mL before measurement. The thermograms were generated by scanning the temperature from 20° C. to 105° C. at a rate of 1° C./min and 60 psi constant pressure was applied during measurement. Four placebo pairs were run before protein samples to generate clean baseline. MicroCal VP-Capillary DSC Automated Analysis software 2.0 was used for data analysis. The T_{m onset} was defined as the temperature where specific heat (Cp) reached 2% of the maximum peak value. Each protein sample was also manually fitted to a non-2 state model to calculate Tₘ values. During the model fitting, peaks were visually selected and fitted until chi square values do not change.

Fluorescence coupled with static light scattering was used to evaluate the onset temperature of tertiary structure unfolding. A UNit® (Unchained labs, Pleasanton, Calif.) system was used to measure the fluorescence and static light scattering simultaneously. During the measurement, ˜8.8 μL protein sample at 1 mg/mL was loaded to the cuvette; the samples were held at 20° C. for 120s and then ramped to 95° C. at the rate of 0.3° C./min. Both fluorescence and static light scattering (at 266 nm) were collected after excitation at 266 nm. After measurement, the data was loaded onto the UNit® analysis software, the raw fluorescence signals were extracted and further processed with excel. The onset of tertiary structure unfolding (Tₒₙₛₑₜ) was defined as the temperature when center of mass (BCM) of the fluorescence emission spectrum (λₘ=π_{λ}f_{λ}/Σ_{λ}f_{λ}) is increased by 0.4% compared to the initial value (the average of the first 5 points). The raw SLS (static light scattering) data were analyzed by the UNit® analysis software, where the onset of aggregation (T_{agg}) is defined as the first temperature at which the first derivative is larger than 0.

### Heparin Column Binding and Hydrophobic Interaction Column Binding

HiTrap Heparin HP Sepharose (GE Healthcare) with a 1 mL capacity and an Agilent 1100 (Santa Clara, Calif.) system were used for evaluating the relative heparin binding affinity of the mAbs. In the experiment, 40 μg proteins were injected to the column and eluted using a linear gradient of 0 to 1M NaCl at 20 mM potassium phosphate, pH 7.0 with 214 nm UV detector and the flow rate was 1 mL/min.

The Tosho NPR Butyl column (San Francisco, Calif.) and an Agilent 1100 (Santa Clara, Calif.) system were used to evaluate the relative hydrophobic interaction potential of the mAbs. Stock solutions of each mAb were diluted to 0.5 mg/mL with 50 mM Potassium Phosphate, pH 6.7, 1M ammonium sulfate. In the experiment, 5 μg proteins were injected to the column and eluted using a linear gradient of 1 to 0 M ammonium sulfate at 50 mM potassium phosphate, pH 6.7 with 214 nm UV detector and the flow rate was 0.5 mL/min.

The elution time of each sample was recorded to evaluate the relative heparin interaction potential (HpnIP %) or hydrophobicity interaction potential (HIP %):

$$HpnIP \% or HIP \% = \frac{{T}_{i} - {T}_{o}}{{T}_{e} - {T}_{o}} \times 100 \qquad Equation 1$$

Where Tᵢ is the elution time of sample, Tₒ is the column equilibrium time before the gradient, Tₑ is the time for the end of the gradient.

Sprague Dawley Rat Pharmacokinetic Studies Sprague Dawley rats were obtained from The Jackson Laboratory (Bar Harbor, Me.). All rats were treatment-naive male between the ages of 8 to 11 weeks with an average weight of 0.3 kg (+/−0.05 kg). PK studies were conducted at Covance (Madison, Wis.) and were designed and executed within accordance of the Animal Use Protocol (AUP) and adherence to the Covance Institutional Animal Care and Use Committee (IACUC) regulations. The mAbs were dosed both IV and SC at 1 mg/kg with a dose volume of 1 mL/kg (dose prepared in PBS pH 7.4). A dose of 1 mg/kg was selected as no target mediated drug disposition (TMDD) was expected in the rodents for any of the antibodies. Blood samples were collected from the jugular vein at 0.083, 1, 6, 12, 24, 48, 72, 96, 120, 168, 240 and 336 hours after dose administration in replicates of 2 or 3 for each mAb. The blood samples were allowed to clot at ambient temperature prior to centrifugation to obtain serum.

### Cynomolgus Monkey Pharmacokinetic Studies

All monkeys were between the ages of 2- to 3 years old with an average weight of 3 kg (+/−0.5 kg). PK studies were conducted at Covance (Madison, Wis.) and were designed and executed within accordance of the Animal Use Protocol (AUP) and adherence to the Covance IACUC regulations. The Platform 1 and 3 mAbs were dosed both IV and SC at 1 or 5 mg/kg with a dose volume of 1 mL/kg (dose prepared in PBS pH 7.4). These doses were selected because there was no TMDD expected in the monkeys for any of the antibodies and anticipated to be in the linear PK range for both platforms allowing for non-target mediated PK parameter estimates across doses and routes. Blood samples were collected from the femoral vein at 1, 6, 12, 24, 48, 72, 96, 168, 240, 336, 432, 504, 600 and 672 hours after dose administration in replicates of 2 for each mAb. The blood samples were allowed to clot at ambient temperature prior to centrifugation to obtain serum. Platform 2 was not evaluated in cynomolgus monkeys due to an expected TMDD that would affect PK.

### Bioanalytical Assays and Pharmacokinetic Data Analysis

Concentrations of the mAbs in Sprague Dawley rats or cynomolgus monkey serum were determined using anti-human IgG or anti-human kappa ELISAs for each of the molecules. In brief, each well of a microtiter plate was coated with either goat anti-human IgG (Jackson ImmunoResearch Laboratories, Inc., West Grove, Pa.) or goat anti-human kappa antibody (Southern Biotech, Birmingham, Ala.). After sample pretreatment of a 1:10 minimum required dilution, washing and blocking, all the standards, control samples, and study samples were added to the plates, then incubated for one hour at room temperature. After washing, the bound molecules were detected with a horseradish peroxidase conjugated mouse anti-human IgG (Fc) antibody (Southern Biotech, Birmingham, Ala.) via TMB Microwell Peroxidase Substrate System (KPL, Gaithersburg, Md.) for a colorimetric response. Plates were read at 450-493 nm with a reference standard of 630 nm. Concentrations from plasma or serum samples were determined from a standard curve prepared with known amounts of the antibody dosed in the measured samples. The concentration of study samples from each mAb were determined by interpolation from a standard curve using a 4/5-parameter logistic curve fit with 1/y² response weighting using Watson LIMS software version 7.4 (Thermo Scientific Inc. Waltham, Mass. USA). The standard curve range for the Platform 1 mAbs ranged from 8 to 500 ng/mL, and the lower limit of quantitation (LLOQ) was defined as 15 ng/mL. The standard curve range for the Platform 2 and 3 mAbs were from 4 to 384 ng/mL, and the lower limit of quantitation (LLOQ) was defined as 8 ng/mL.

Pharmacokinetic parameters were calculated using the WinNonlin Professional (Version 3.2) software package (Pharsight Corporation, Mountain View, Calif.). Serum concentration-time data were calculated using a model-independent approach based on the statistical moment theory. The parameters calculated included the maximum serum concentration (Cₘₐₓ), area under the curve (AUC_{0-∞}), clearance (CL), elimination half-life (t_{1/2}) and rate of absorption (kₐ).

### ¹²⁵I-mAb Preparation and Subcutaneous Tissue Association Quantification in Cynomolgus Monkeys

The Platform 1 and a subset of the Platform 3 mAbs were radiolabeled with ¹²⁵I to monitor the percent loss from the subcutaneous site of injection in cynomolgus monkeys. Radio-iodination (¹²⁵I) of mAbs for percent subcutaneous tissue bound calculations was performed using the succinimidyl iodobenzoate (SIB) iodination method. Briefly, 2-3 mCi of Na ¹²⁵I (Perkin-Elmer, Billerica, Mass.) was reacted with 5-8 μg N-succinimidyl-3-(tri-n-butylstannyl) benzoate (American Advanced Scientific, College Station, Tex.) to generate ¹²⁵I SIB, which in turn was reacted with 1-2 mg of each test mAb, essentially as described. The labeled proteins were purified by gel filtration over OD-10 desalting columns (GE Healthcare, Pscataway, N.J.) to remove unconjugated ¹²⁵I SIB and protein concentrations verified by UV spectroscopy. Dosing solutions were prepared by mixing unlabeled mAbs with the corresponding ¹²⁵I-mAb to a final concentration of 1 mg/ml in buffer. The radioactive specific activity of the dosing solutions was an average of 0.1 mCi/mg that utilized a tissue puncture sampling approach. Radiochemical purity of dosing solutions was characterized by trichloroacetic acid (TCA; Sigma-Aldrich, S. Louis, Mo.) precipitation and size-exclusion HPLC using an Agilent Bio SEC-3 column (Gilent Technologies, Santa Clara, XA). The percentage of free ¹²⁵I was less than 1% in all dosing solutions preparations.

All cynomolgus monkeys were treatment males between the ages of 2 to 3 years old with an average weight of 3 kg (+/−0.5 kg). Studies conducted at Covance (Madison, Wis.) and were designed and executed within accordance of the AUP and adherence to the Covance IACUC regulations. The Platform 2 mAbs were not evaluated due to expected TMDD. ¹²⁵I labeled Platform 1 and 3 mabs were administered SC in the thoracic region at 0.1 mg/kg/site with a dose volume of 300 μL per site of injection (dose prepared in PBS pH 7.4). Two sets of four monkeys were administered ¹²⁵I labeled mAbs 1P and 1RE or ¹²⁵I labeled mAbs 3P and 3RE1 each at pre-determined and distinctly isolated injection sites each for antibody for up to six administration sites per animal.

For quantification of the loss of mAb from the SC tissue administration site, each site of ¹²⁵I labeled mAb administration underwent a skin punch biopsies of 8 mm at a specified post dose time. One skin punch biopsy represented one site of administration at a pre-determined post dose time of 1 and 6 hours post dose. Skin biopsy punctures were weighed directly following collection. Each skin puncture count was measured using a gamma counter (Wallac Wizard 1480, Perkin Elmer, Waltham, Mass.) and percent bound to the SC tissue was calculated. The 1 hour post dose time point skin punctures radioactive count for each mAb was considered 100% bound for data normalization purposes. The 6 hour post dose collected radioactivity data were compared reported as a fraction of the percent bound relative to the 1 hour post dose time point for calculation, data processing and loss of mAb from the SC site reporting over time.

### Results

### Description of the IgG Molecules

In the present evaluation, several humanized IgG molecules, across three platforms, were tested to characterize the connectivity between mAb physiochemical properties and PK parameters following SC administration. Each antibody platform was developed against a different undisclosed target. Within each platform two or three mAbs were characterized; a parental molecule and one or two re-engineered mAb that was designed to have physiochemical property changes distinct from the parent. Platform 1 is comprised of two humanized IgG₁ molecules including, the parental (P) mAb 1P, which comprises three LCDRs having sequences in SEQ ID NOs: 1, 2, 3, respectively, and three HCDRs having sequences in SEQ ID NOs: 4, 5, 6, respectively; and the re-engineered (RE) mAb 1RE, which comprises three LCDRs having sequences in SEQ ID NOs: 1, 2, 3, respectively, and three HCDRs having sequences in SEQ ID NOs: 7, 5, 8, respectively. Platforms 2 and 3 each consist of humanized IgG₄ constructs. The molecules in Platform 2 are the parental mAb 2P, which comprises three LCDRs having sequences in SEQ ID NOs: 9, 10, 11, respectively, and three HCDRs having sequences in SEQ ID NOs: 12, 13, 14, respectively; and the re-engineered mAb 2RE, which comprises three LCDRs having sequences in SEQ ID NOs: 9, 15, 11, respectively, and three HCDRs having sequences in SEQ ID NOs: 12, 16, 14, respectively. Platform 3, consists of the parental mAb 3P, which comprises three LCDRs having sequences in SEQ ID NOs: 17, 18, 19, respectively, and three HCDRs having sequences in SEQ ID NOs: 20, 21, 22, respectively; the re-engineered mAb 3RE1, which comprises three LCDRs having sequences in SEQ ID NOs: 23, 24, 25, respectively, and three HCDRs having sequences in SEQ ID NOs: 26, 27, 28, respectively; and the re-engineered mAb 3RE2, which comprises three LCDRs having sequences in SEQ ID NOs: 29, 30, 31, respectively; and three HCDRs having sequences in SEQ ID NOs: 32, 33, 28, respectively (see Tables 1 and 2).

The CDR sequences are aligned based on Kabat numbering; and the CDRs are defined by a hybrid of Kabat and Chothia. The key re-engineered CDR residues of the mAbs are bolded and underlined, which include one or more of the following amino acid residues: lysine (K) at position 24 of LCDR1; leucine (L) at position 54 of LCDR2; aspartic acid (D) or glutamic acid (E) at position 55 of LCDR2; serine (S) or threonine (T) at position 56 of LCDR2; phenylalanine (F) at position 96 of LCDR3; and glutamic acid (E) at position 61 of HCDR2 (all residues numbered based on Kabat numbering).

Analysis of the CDR sequences of the reengineered mAbs reveals they comprise one or more of the following common residues: alanine (A) or serine (S) at position 25 of LCDR1; serine (S) at position 26 of LCDR1; serine (S) or threonine (T) at position 52 of LCDR2; glutamine (Q) or valine (V) at position 89 of LCDR3; glutamine (Q) at position 90 of LCDR3; proline (P) at position 95 of LCDR3; threonine (T) at position 97 of LCDR3; glycine (G) at position 26 of HCDR1; tyrosine (Y) at position 27 of HCDR1; phenylalanine (F) at position 29 of HCDR1; threonine (T) at position 30 of HCDR1; lysine (K) at position 62 of HCDR2; and glycine (G) at position 65 of HCDR2.

TABLE 1
Light Chain CDR Sequences of the mAbs

| | LCDR1 | LCDR2 | LCDR3 |
|---|---|---|---|
| Kabat Numbering | 24 34 | 50 56 | 89 97 |
| IP | KSSQSLLYSRGKTYLN | AVSKLDS | VQGTHYPFT |
| | (SEQ ID NO: 1) | (SEQ ID NO: 2) | (SEQ ID NO: 3) |
| IRE | KSSQSLLYSRGKTYLN | AVSKLDS | VQGTHYPFT |
| | (SEQ ID NO: 1) | (SEQ ID NO: 2) | (SEQ ID NO: 3) |
| 2P | RASKSIS-----KYTA | AGSKRHW | QQHNEYPYT |
| | (SEQ ID NO: 9) | (SEQ ID NO: 10) | (SEQ ID NO: 11) |
| 2RE | RASKSISKYTA | AGSKLHW | QQHNEYPYT |
| | (SEQ ID NO: 9) | (SEQ ID NO: 15) | (SEQ ID NO: 11) |
| 3P | RSSQSLLISGGKTYLN | LVSKLDQ | WQGTYFPLT |
| | (SEQ ID NO: 17) | (SEQ ID NO: 18) | (SEQ ID NO: 19) |
| 3RE1 | KASDHIG-----KFLT | GATSKLT | QQYWSTPFT |
| | (SEQ ID NO: 23) | (SEQ ID NO: 24) | (SEQ ID NO: 25) |
| 3RE2 | KASDHIL-----KFLT | GATSLET | QMYWSTPFT |
| | (SEQ ID NO: 29) | (SEQ ID NO: 30) | (SEQ ID NO: 31) |

TABLE 2
Heavy Chain CDR Sequences of the mAbs

| | HCDR1 | HCDR2 | HCDR3 |
|---|---|---|---|
| Kabat Numbering | 26 35 | 50 65 | 95 102 |
| IP | GYTFTRYYIN | INPGSGNTKYNEKFKG | ----EGTTVY |
| | (SEQ ID NO: 4) | (SEQ ID NO: 5) | (SEQ ID NO: 6) |
| IRE | GYTFTDYYIN | INPGSGNTKYNEKFKG | ----EGETVY |
| | (SEQ ID NO: 7) | (SEQ ID NO: 5) | (SEQ ID NO: 8) |
| 2P | GYAFTSFLIE | SNPRTGRTKYKSKFRG | ----EFFD-Y |
| | (SEQ ID NO: 12) | (SEQ ID NO: 13) | (SEQ ID NO: 14) |
| 2RE | GYAFTSFLIE | SNPRTGGRKYKEKFRG | ----EFFD-Y |
| | (SEQ ID NO: 12) | (SEQ ID NO: 16) | (SEQ ID NO: 14) |
| 3P | GKTFWSYGIN | IYIGTGYTEPNPKYKG | IGGYYGNFDQ |
| | (SEQ ID NO: 20) | (SEQ ID NO: 21) | (SEQ ID NO: 22) |
| 3RE1 | GYPFTRYVMH | INPYNDGVNYNEKFKG | ----NWDTGL |
| | (SEQ ID NO: 26) | (SEQ ID NO: 27) | (SEQ ID NO: 28) |
| 3RE2 | GYKFTRYVMH | INPYNDGTNYNEKFKG | ----NWDTGL |
| | (SEQ ID NO: 32) | (SEQ ID NO: 33) | (SEQ ID NO: 28) |

Overall, the three platforms were leveraged to dissect the role of charge and hydrophobicity in mAb kinetics following SC administration. The Platform 1 molecules were leveraged to understand the role of charge; the Platform 2 molecules had components of both charge and hydrophobicity; the Platform 3 molecules were predominantly influenced by hydrophobicity differences. Table 3 lists the constructs and a high-level summary of their qualitative biophysical properties.

TABLE 3
General Description of the mAbs*

| | | | | Hydrophobic | | | |
|---|---|---|---|---|---|---|---|
| | | | Charge Based | Based | | | TMDD in |
| | | mAb | Interaction | Interaction | | TMDD | Cynomolgus |
| Platform | mAb | Isotype | Potential | Potential | T_{agg}* | in Rats\hat{ } | Monkeys\hat{ } |
| 1 | 1P | IgG1 | +++ | − | − | − | − |
| (Target | 1RE | IgG1 | ++ | − | − | − | − |
| 1 mAbs) | | | | | | | |
| 2 | 2P | IgG4 | ++++++++ | ++ | ++ | − | + |
| (Target | 2RE | IgG4 | ++++ | ++ | + | − | + |
| 2 mAbs) | | | | | | | |
| 3 | 3P | IgG4 | ++ | +++ | ++ | − | − |
| (Target | 3RE1 | IgG4 | + | + | − | − | − |
| 3 mAbs) | 3RE2 | IgG4 | +++ | ++ | − | − | − |

*All the molecules are humanized IgGs. The ‘+’ and ‘−’ signs indicate the presence and absence of a characteristic, respectively. The number of ‘+’ symbols within the charge- and hydrophobic-based interaction potential columns are intended to provide a qualitative perspective of the relative preponderance of each characteristic across and within the mAb platforms. Quantitative values for the charge- and hydrophobic-based interaction potential are in Table 2.
*T_{agg} is the temperature of aggregation onset.
\hat{ }TMDD = target mediated drug disposition

Characterization of the Physiochemical Properties of the mAbs

Table 4 summarizes the physiochemical attributes of the mAbs in each platform via a battery of analyses aimed at understanding the physiochemical profiling connected with the PK and absorption following SC administration. Molecular interactions governed by hydrophobic and charge-based mechanisms were evaluated using multiple orthogonal approaches. In addition, molecules were also assessed for overall thermal stability, as well as, their aggregation potential.

The global molecule hydrophobicity was determined using a chromatographic HIC (hydrophobic interaction column)-based method. The data were expressed as a relative hydrophobicity interaction percentage for each of the mAbs to allow for comparisons both within and across the three mAb platforms; larger hydrophobicity interaction percent (HIP) values indicate an increased affinity for the HIC matrix. The Platform 1 molecules show similar and relatively low HIP values; the HIP for mAb 1P and mAb 1RE were 1.3% and 0.7%, respectively. In contrast, both the Platform 2 and 3 molecules showed ˜10- to ˜100-times higher HIP values than the Platform 1 mAbs (Table 4). The Platform 2 constructs showed similar HIP values for mAb 2P and mAb 2RE of ˜16% and ˜20%, respectively. Platform 3 mAbs had the widest diversity of HIP with mAb 3P, 3RE1 and 3RE2 displaying values of ˜100%, ˜12% and ˜17% respectively.

TABLE 4
Biophysical and FcRn Binding Properties of the mAbs*

| | | FcRn | | | Zeta | | | | | | |
|---|---|---|---|---|---|---|---|---|---|---|---|
| | | K_{d} (nM) | HpnIP | HIP | Potential | | | T_{m onset} | Tₘ (° C.) | | |
| Platform | mAb | at pH 6 | (%) | (%) | (mV) | pI | T_{agg} (° C.) | (° C.) | C_{H}2 | C_{H}3 | Fab |
| 1 | 1P | 105 ± 5 | 51.8 | 1.3 | 6.2 ± 1.4 | 8.8 | 64.2 ± 0.5 | 61.1 | 67 | 83 | 70 |
| | 1RE | 98 ± 10 | 28.3 | 0.7 | 9.5 ± 0.2 | 9.1 | 64.5 ± 0.2 | 61.7 | 70 | 83 | 70 |
| 2 | 2P | 93 ± 4 | 100 | 16.0 | 10.5 ± 1.4 | 9.2 | 52.4 ± 2.1 | 56.6 | 69 | 72 | 76 |
| | 2RE | 121 ± 15 | 58.1 | 20.1 | 9.7 ± 1.9 | 9.5 | 59.6 ± 1.1 | 62.0 | 69 | 73 | 76 |
| 3 | 3P | 101 ± 7 | 31.6 | 100 | 9.4 ± 0.8 | 8.3 | 55.6 ± 2.3 | 58.9 | 69 | 73 | 68 |
| | 3RE1 | 113 ± 4 | 18.6 | 11.7 | 9.7 ± 0.6 | 8.5 | 63.9 ± 2.7 | 62.2 | 69 | 73 | 78 |
| | 3RE2 | 97 ± 8 | 45.9 | 16.7 | 6.24 ± 0.60 | 8.5 | 61.4 ± 0.6 | 62.0 | 70 | 74 | 78 |

*FcRn K_{d} represents cynomolgus monkey FcRn binding affinity at pH 6. HpnIP is the relative heparin binding interaction potential. HIP is the relative hydrophobic interaction potential. pI is the isoelectric point. T_{agg} is the temperature of aggregation onset. T_{m onset} is the onset of tertiary structure unfolding.

The charge of the mAbs was evaluated using multiple orthogonal approaches. Global mAb surface charge was assessed through determining the pI and zeta potential, whereas, local surface charge was determined through heparin binding interactions. The pI values were determined using capillary isoelectrophoresis. The results indicated some subtle differences in the pI of molecules when compared within and across each platform (<0.2 units) (Table 4). The Platform 1 mAbs 1P and 1RE had pI values of 8.8 and 9.1, respectively; Platform 2 mAbs 2P and mAb 2RE molecules had pI values of 9.2 and 9.0, respectively; Platform 3 mAbs 3P, 3RE1 and 3RE2 constructs had pI values of 8.3, 8.5 and 8.5, respectively (Table 4). The zeta potential of the mAbs was determined using electrophoretic light scattering. The zeta potential of the molecules trended to be similar with the exception of the non-significantly lower and higher potentials observed for mAbs 1P and 2P, respectfully (Table 4). The interaction of the mAbs with heparin was evaluated using a heparin coated matrix packed into a column. Heparin was selected since it is found in abundance on the SC capillaries. Previously, it was reported the interaction of molecules with heparin using heparin-coated plates (Datta-Mannan A, et al., mAbs 2015; 7:483-93; Datta-Mannan A, et al., mAbs 2015; 7:1084-93). This method was dependent on the detection of heparin bound mAbs using an antibody based detection. Differences in the cross-reactivity of the mAbs with the detection antibody created challenges in the quantitative comparison of the heparin binding across molecules (data not shown). Thus, all three mAb platforms were evaluated using a heparin column and UV-VIS based detection to allow for adequate comparisons of the heparin interaction across molecules. The data were expressed as a relative heparin interaction percentage (HpnIP) for each of the mAbs to allow for comparisons both within and across the three mAb platforms; larger heparin interaction percent (HpnIP) values indicate an increased affinity for the heparin matrix. The Platform 1 molecules show an ˜1.9-fold difference HpnIP values; the HpnIP for mAbs 1P and mAb 1RE were ˜52% and ˜28%, respectively. The Platform 2 molecules showed ˜1.7-fold differences with mAb 2P and mAb 2RE displaying ˜100% and ˜58% HpnIP values, respectively. The Platform 3 mAbs had HpnIP values with mAb 3P, 3RE1 and 3RE2 ˜32%, ˜19%, and ˜46%, respectively.

The Tₘ of the mAbs was determined using differential scanning calorimetry (DSC). Within the three platforms, a higher onset of melting temperature (T_{m onset}) was observed for mAb 1RE relative to 1P, mAb 2RE relative to 2P and mAb 3RE1 and 3RE2 each relative to 3P (Table 4). Differences in Tₘ values were observed in Fab regions within Platform 3, the C_{H}2 domains of the Platform 1 molecules and the C_{H}3 of the Platform 2 mAbs (Table 4). In addition to DSC, simultaneous static light scattering (SLS) and fluorescence spectroscopy were used to monitor aggregation (T_{agg}) and the onset temperature (T_{m onset}) of tertiary structure unfolding. The parabolic nature of protein unfolding free energy dependence on temperature determines that at high temperature protein will readily unfold. During thermal ramping from moderate temperature, protein will be partially unfolded at certain point and consequently will drive further intermolecular interactions and finally aggregation. SLS is sensitive to trace amount of aggregates and well suited for measuring aggregation onset. The unfolding event in the meantime will trigger red shifting of the fluorescence spectrum due to the exposure of aromatic amino acid and in this case tryptophan. Therefore, simultaneous SLS and fluorescence spectroscopy is able to capture the aggregation propensity and conformational stability at the same time during thermal ramping. The results are reported in Table 4. The temperature of aggregation onset (T_{agg}) of the Platform 1 mAbs 1P and mAb 1RE were 64.2° C. and 64.5° C., respectively. The T_{agg} increased in the Platform 2 mAbs from 52.4° C. for mAb 2P to 59.6° C. for mAb 2RE. In the case of Platform 3, the T_{agg} increased from 55.6° C. for mAb 3P to 63.9° C. for mAb 3RE1 and 61.4° C. for mAb 3RE2. In the meantime, the unfolding onset (T_{m onset}) of the Platform 2 mAbs improved from 57.6° C. to 62.0° C. for mAbs 2P and 2RE, respectfully, as well as, for the Platform 3 mAbs from 58.9° C. for mAb 3P to 62.2° C. and 62.0° C. for mAbs 3E1 and 3RE2, respectfully, after re-engineering (Table 4).

The binding affinities of mAbs with immobilized cFcRn was measured using previously reported surface plasmon resonance approaches (Datta-Mannan A, Drug metabolism and disposition: the biological fate of chemicals 2012; 40:1545-55). The binding affinity (K_{d}) of the mAbs for cFcRn at pH 6.0 ranged from −93 to 121 nM across the three mAb platforms. No direct binding to cFcRn at pH 7.4 was detected for any of the mAbs (data not shown).

Evaluation of the Pharmacokinetics of the mAbs in Rats

The PK of the constructs was evaluated following a single IV or SC administration to rats due to the ability to serially sample individual animals over the duration of the in vivo study. In addition, in rats none of the molecules has a target mediated component to their clearance (i.e. target mediated drug disposition or TMDD) either due to low endogenous antigen concentrations or the lack of the cross-reactivity of the antibodies with rat antigen; thus, the inherent influence of the physiochemical parameters of the mAbs on PK could be evaluated in the absence of the kinetic complexities associated with TMDD.

The PK parameters following a single 1 mg/kg IV or SC administration of the mAbs are reported in Table 5. Overall, the apparent clearance (CL/F) (and intrinsic clearance in the case of the IV administration), rate of SC absorption (kₐ) and SC bioavailability of the re-engineered mAbs in each platform (1RE, 2RE and 3RE1 and 3RE2) improved relative to their respective parental mAbs (1P, 2P and 3P, respectively) (Table 5).

TABLE 5
Rat Pharmacokinetic Parameters of the mAbs

| | | | Dose\hat{ } | Cₘₐₓ | Tₘₐₓ | AUC_{0-inf} | CL or CL/F | T_{1/2} | kₐ ^{A} | |
|---|---|---|---|---|---|---|---|---|---|---|
| Platform | mAb | Route | (mg/kg) | (μg/mL) | (hr) | (hr*μg/mL) | (mL/hr/kg) | (hr) | (1/hr) | % F |
| 1 | 1P | IV | 1 | 22.4 ± 3.7 | NA | 1345.5 ± 252.5 | 0.62 ± 0.22 | 163 ± 79 | NA | NA |
| | | SC | 1 | 5.0 ± 1.0 | 96 ± 0 | 1294.9 ± 134.9 | 0.78 ± 0.08 | 133 ± 28 | 0.009 ± 0.001 | 108 ± 17 |
| | 1RE | IV | 1 | 32 ± 3.8 | NA | 1513 ± 229 | 0.37 ± 0.03 | 96 ± 9 | NA | NA |
| | | SC | 1 | 10.0 ± 1 | 64 ± 37 | 2176 ± 159 | 0.3 ± 0.1 | 221 ± 101 | 0.021 ± 0.012 | 144 ± 11 |
| 2 | 2P | IV | 1 | 10.6 ± 1.3 | NA | 210 ± 52 | 4.5 ± 0.98 | 117 ± 9 | NA | NA |
| | | SC | 1 | 0.2 ± 0 | 72 ± 72 | 39 ± 25 | 6.4 ± 7.0 | 1375.2 ± 1550.0 | 0.036 ± 0.006 | 25 ± 4^{#} |
| | 2RE | IV | 1 | 21.7 ± 0.8 | NA | 928 ± 49 | 0.97 ± 0.07 | 108 ± 9 | NA | NA |
| | | SC | 1 | 3.0 ± 0.3 | 104 ± 55 | 564 ± 152 | 1.5 ± 0.8 | 158 ± 81 | 0.012 ± 0.003 | 61 ± 16 |
| 3 | 3P | IV | 1 | 36.9 ± 1.7 | NA | 2088 ± 224 | 0.48 ± 0.06 | 129 ± 14.2 | NA | NA |
| | | SC | 1 | 3.0 ± 1.0 | 104 ± 60 | 699 ± 168 | 0.1 ± 0.2 | 210 ± 62 | 0.038 ± 0.016 | 33 ± 8 |
| | 3RE1 | IV | 1 | 47.5 ± 6.8 | NA | 3200 ± 121 | 0.21 ± 0.04 | 174 ± 21 | NA | NA |
| | | SC | 1 | 9.5 ± 1.0 | 120 ± 0 | 1816 ± 490 | 0.5 ± 0.2 | 154 ± 32 | 0.013 ± 0.004 | 57 ± 17 |
| | 3RE2 | IV | 1 | 33.5 ± 1.7 | NA | 2856 ± 237\hat{ }\hat{ }\hat{ } | 0.21 ± 0.02 | 284 ± 76 | NA | NA |
| | | SC | 1 | 7.5 ± 1.0 | 128 ± 97 | 1892 ± 265\hat{ }\hat{ }\hat{ } | 0.21 ± 0.05 | 433.4 ± 29.2 | 0.024 ± 0.01 | 66 ± 5 |

Cₘₐₓ, maximal observed serum concentration; Tₘₐₓ, time of maximal observed serum concentration; AUC_{0-inf}, area under the serum concentration curve from time zero extrapolated to infinite time; CL, clearance following IV administration; CL/F, apparent clearance as a function of bioavailability following SC administration; T_{1/2}, elimination half-life; ka, rate of SC absorption; % F, SC bioavailability.
NA = not applicable.
\hat{ }\hat{ }\hat{ }AUC all reported.
\hat{ }N = 3/timepoint with serial sampling unless otherwise noted.
^{#}N = 2 for % F reporting only (standard deviation for illustrative purposes only).
^{A} Determined from two compartment pharmacokinetic analyses

Pharmacokinetics of the Platform 1 and 3 Variants mAbs in Cynomolgus Monkeys

The SC space varies in composition across species; thus, as a means to understand if the SC PK findings in rats were meaningful in another species, the PK of a subset of molecules was evaluated in cynomolgus monkeys since this species is commonly utilized for predictions of human antibody PK. The PK in cynomolgus monkeys was evaluated following a single 1 mg/kg IV or SC administration of Platform 1 and 3 mAbs. The Platform 2 molecules were not evaluated due to the known cross-reactivity of the molecules in this group with cynomolgus monkey target that leads to non-linear clearance following IV administration (data not shown) and thus would likely confound the interpretation of SC PK. The PK parameters following a single 1 mg/kg IV or SC administration of the Platform 1 and 3 mAbs are reported in Table 6. Overall, the apparent clearance (CL/F) (and intrinsic clearance in the case of the IV administration), rate of SC absorption (ka) and SC bioavailability of the re-engineered mAbs in each platform (1RE and 3RE1 and 3RE2) improved relative to their respective parental mAbs (1P and 3P, respectively)) (Table 6).

TABLE 6
Cynomolgus Monkey Pharmacokinetic Parameters of the mAbs

| | | | Dose\hat{ } | Cₘₐₓ | | AUC_{0-∞} | CL or CL/F | | | |
|---|---|---|---|---|---|---|---|---|---|---|
| Platform | mAb | Route | (mg/kg) | (μg/mL) | Tₘₐₓ (hr) | (hr*μg/mL) | (mL/hr/kg) | T_{1/2} (hr) | kₐ^{A}(1/hr) | % F |
| 1 | 1P | IV | 1 | 24.0 ± 0.6 | NA | 2910 ± 587 | 0.35 ± 0.08 | 278 ± 76 | NA | NA |
| | | SC | 1 | 4.0 ± 0.7 | 48 ± 24 | 1492 ± 201 | 0.68 ± 0.10 | 248 ± 23 | 0.033 ± 0.006 | 51 ± 7 |
| | 1RE | IV | 1 | 26.9 ± 2.4 | NA | 4264 ± 1242 | 0.25 ± 0.06 | 382 ± 162 | NA | NA |
| | | SC | 1 | 12.6 ± 2.6 | 24 ± 0 | 3187 ± 111 | 0.31 ± 0.01 | 235 ± 53 | 0.021 ± 0.003 | 75 ± 3 |
| 3 | 3P | IV | 1 | 27 ± 1.3 | 0.17 +/− 0.12 | 1832 ± 231 | 0.56 ± 0.7 | 271.8 ± 55.9 | NA | NA |
| | | SC | 1 | 3.4 ± 1.3 | 60 +/− 17 | 837 ± 452 | 1.4 ± 0.8 | 214.5 ± 102.9 | 0.094 ± 0.002 | 46 ± 6 |
| | 3RE1 | IV | 1 | 25.54 ± 3.6 | NA | 3473 +/− 2048 | 0.35 ± 0.21 | 213.2 ± 141.2 | NA | NA |
| | | SC | 1 | 7.92 ± 0.4 | 132 ± 50.9 | 3907 ± 2406 | 0.32 ± 0.19 | 337.8 ± 48.2 | 0.049 ± 0.003 | 100 ± 8 |
| | 3RE2 | IV | 1\hat{ }\hat{ } | 25.6 ± 3.8 | NA | 856 ± 43.0\hat{ }\hat{ }\hat{ } | 1.17 ± 0.06 | 113.5 ± 2.8 | NA | NA |
| | | SC | 1 | 4.5 ± 2.4 | 15 ± 12.7 | 283 ± 52\hat{ }\hat{ }\hat{ } | 3.35 ± 0.07 | 42.3 ± 18.2 | 0.207 ± 0.002 | 33^{#} ± 6 |

Cₘₐₓ, maximal observed serum concentration; Tₘₐₓ, time of maximal observed serum concentration; AUC_{0-∞}, area under the serum concentration curve from time zero extrapolated to infinite time; CL, clearance following IV administration; CL/F, apparent clearance as a function of bioavailability following SC administration; T_{1/2}, elimination half-life; kₐ, rate of SC absorption; % F, SC bioavailability.
NA = not applicable.
\hat{ }N = 3 cynomolgus monkeys/time point.
^{#}N = 2 for % F reporting only (standard deviation for illustrative purposes only). All PK parameters were determined from non-compartmental pharmacokinetic analyses unless otherwise noted.
\hat{ }\hat{ }Dose normalized from 5 mg/kg to 1 mg/kg assuming dose proportionality.
\hat{ }\hat{ }\hat{ }AUC all reported
^{A}Determined from two compartment pharmacokinetic analyses

Evaluations of the Relative SC Tissue Association for Platform 1 and 3 Variants mAbs in Cynomolgus Monkeys

The PK studies suggested a reduction in the amount of the parental mAbs 1P and 3P absorbed into the systemic circulation following SC administration relative to their re-engineered counterparts, mAbs 1RE and 3RE1, respectfully. As a means to begin to dissect if the lower extent and rate of absorption of the parental mAbs was due to increased residence time within the SC injection site, the exposure of SC tissue association at the injection site at 6 hours post administration for the Platform 1 and 3 molecules was assessed in cynomolgus monkeys. The tissue association for the Platform 1 mAbs shows ˜30% increased retention of the parental mAb 1P within the SC tissue at 6 hours post-dose relative to the re-engineered mAb 1RE. In the case of Platform 3, the parental mAb 3P has an ˜2-fold increased retention/association at 6 hours post SC administration compared to the re-engineered mAb 3RE1. Relative to the re-engineered mAbs 1RE and 3RE1, the counterpart parental mAbs 1P and 3P, respectfully, had shown reduced SC bioavailability and decreased kₐ values. Taken together with the SC tissue association findings, the data indicate increased SC tissue association at the injection site reduces mAb exposure.

The impact of multiple mAb physiochemical factors (charge, hydrophobicity, aggregation potential and thermal stability) on the rate and extent of SC absorption of humanized mAbs was tested in rats and cynomolgus monkeys. The clearance of these tested mAbs was not a consequence of target interactions (TMDD) since the kinetics of the mAbs were assessed in a species (rat, cynomolgus monkey or both) in which there were insignificant concentrations of antigen present to influence the clearance. The PK was also unrelated to aberrant FcRn binding as the mAbs showed receptor binding affinities at pH 6 in the range reported for molecules with well-behaved kinetics, as well as, no direct FcRn interactions at neutral pH (Table 4). Thus, the focus was on understanding the role of the aforementioned physiochemical parameters in the context of the SC space/anatomy and composition. Importantly, using this approach, several physiochemical properties are found critical with regard to their influence on mAb kinetics following SC administration and that some of these (e.g., T_{m onset} and T_{agg}) are unique with regard to having increased connectivity with SC relative to IV administration across species.

The reduced SC bioavailability, slowed rate of SC absorption and increased clearance of parental mAbs across the three platforms relative to their re-engineered counterparts in rats and cynomolgus monkeys, herein, point towards taking into account inherent mAb charge, hydrophobicity, aggregation potential, and thermal stability as a means to engineer these molecules for improved in vivo SC absorption rate and bioavailability. The relative contribution of each of these factors underlies the basic in vivo characteristics of an antibody and heavily influences strategies aimed at optimizing the PK properties of mAbs (Chaparro-Riggers J, et al. The Journal of biological chemistry 2012; 287:11090-7; Datta-Mannan A, and Wroblewski V J. Drug metabolism and disposition: the biological fate of chemicals 2014; 42:1867-72; Igawa T, et al. Nature biotechnology 2010; 28:1203-7; Yeung Y A, et al. Cancer Res 2010; 70:3269-77). In this study, the combination of high local positive charge (as measured by heparin interactions), increased hydrophobic interaction potential and low thermal stability leading to increased aggregation potential had the largest negative effect on the rate of SC absorption, apparent clearance and bioavailability as evident from the PK findings for mAb 2P in rats compared to the other antibodies within and across Platforms. Thus, it is recommended to consider these factors for improved in vivo performance following SC administration. Given the increased compliance and convenience benefits of SC administration for patients, dissecting factors influencing the SC disposition of mAbs will further extend the drug-ability of mAb-based therapeutics and improve patient outcomes and experience.

Considerable insight was gained from the comparison of the physiochemical characterization of the mAbs with regard to global (pI and zeta potential) and local surface charge (HpnIP value) assessment. Some similarities and a number of differences in the sensitivity of the local and global charge findings across the three Platforms were observed in this comparison. In the studies for the global assessment of mAb surface charge, the pI values of the mAbs ranged from 8.2 to 9.4; however, within a Platform, the pI values were similar and showed no more than a marginal 0.3 pI unit shift. This was the case even for molecule pairs in each Platform which showed >2-fold differences in HpnIP values, which gives additional insight into the assessment of local surface charge being a more sensitive measure of potential charge based interactions than the pI. It is worth noting, that given the overall large molecular weight of mAbs, the few resides altered across mAbs within a Platform and well-ordered tertiary structure of antibodies, the modest changes in a global measure of charge, such as pI, within a given Platform is not surprising. Similar to the pI observation, changes in zeta potential (which is also a global measure of charge) across the molecules were also marginal and thus indicated similar overall or global surface net charge of the mAbs. These global assessments of surface charge were difficult to fully interpret/connect with the SC PK findings. Previous reports have suggested some mixed finding with regard to the value of assessing pI or other global measures of mAb surface charge and their connectivity to mAb kinetics (Igawa T, et al. Protein Eng Des Sel 2010; 23:385-92; Li B, et al. mAbs 2014; 6:1255-64; Sampei Z, et al. PloS one 2013; 8:e57479; Datta-Mannan A, et al. mAbs 2015; 7:483-93). In the current study, the largest difference in charge-based interactions within and across mAb Platforms was observed with HpnIP, which is more sensitive in detecting local charge patches compared to global surface charge assessments (i.e. pI or zeta potential measures). All the molecules showed some level of charge-based interaction in the heparin column interaction assay indicative of the potential to have non-specific binding (NSB) for negatively charged in vivo matrices such as the SC space. The reported finding indicates that the local position of charge display (i.e. in a solvent accessible area such as the CDR) is an impactful aspect which maybe under-represented in the overall pI or zeta potential determination and better characterized via HpnIP when considering mAb engineering approaches for PK connectivity attributes.

In addition, to the component of charge-based driven NSB potential influencing PK, the physiochemical characterization for the three mAb Platforms indicates the hydrophobic interactions also impact kinetics. These are potentially more important in the context of in vivo interactions within the SC space where compositionally there are fat lobules, adipocytes, collagen and other connective tissues which likely favor non-covalent hydrophobic interactions. Platforms 2 and 3 (HIP range of ˜16-100%) had much larger inherent HIP than Platform 1 (HIP values of ˜1%), suggesting the potential for a combination of both charge- and hydrophobic-related interactions with varying degrees impacting the in vivo performance of these molecules. Interestingly, the inherent differences in hydrophobic-based interactions showed some connectivity with the onset of thermally induced tertiary structure unfolding and aggregation potential across the three mAb Platforms. The genesis of this observation is the aggregation onset (T_{agg}) across the three Platforms presented here. In the case of the Platform 1 mAbs, the T_{agg} of mAb 1P (parental with reduced SC % F) and mAb 1RE (re-reengineered mAb with higher SC % F); these mAbs predominantly show differences in their charge compared to hydrophobic based interactions. In contrast, the T_{agg} is improved for both the Platform 2 mAbs 2P and mAb 2RE (from 52.4° C. to 59.6° C., respectively) and Platform 3 mAbs 3P, 3RE1 and 3RE2 (from 55.6° C. to 63.9° C. and 61.4, respectively); however, unlike the Platform 1 mAbs, the Platform 2 and 3 molecules displayed increasing differences in their hydrophobic compared to charge based interactions. The improved T_{agg} observed in Platform 2 with mAb 2RE relative to mAb 2P, as well as, in Platform 3 with the relative rank order of T_{agg} improvement of mAb 3RE1>mAb 3RE2>mAb 3P combined, indicate molecules with increased HIP are more sensitive to unfolding. This may be due to a propensity for attempting to bury solvent accessible hydrophobic regions through a greater inclination to change conformation via unfolding and thereby result in an increased aggregation. The reduction in surface hydrophobicity led to reduced tendency to aggregate as measured by T_{agg}. The comparable T_{agg} of the Platform 1 mAbs, which were predominately influenced by charge-based interactions (as observed in HpnIP), that typically behave in a repulsive manner with regard to self-association or aggregation, also support this hypothesis.

The Platform 1 mAbs were reasonable surrogates for predominantly studying the impact of local charge-related NSB on SC absorption and bioavailability given these molecules showed strong charge-based binding signals and little/no hydrophobic interaction potential (values in the single digit percentage range) in vitro. Since the SC space consists of a milieu of negatively charged GAGs and other proteoglycans, the reduced ka and SC bioavailability of the more solvent exposed positive charge parental mAb 1P in both rats and cynomolgus monkeys, suggests a mechanism whereby mAb 1P has enhanced residence within the SC space, which reduces the rate and extent of mAb 1P being absorbed into the blood circulation compared with the re-engineered lower HpnIP mAb 1RE. This hypothesis is also supported by the observed increased SC tissue association of mAb 1P observed in cynomolgus monkey relative to mAb 1RE. It is possible that reducing the local charge related HpnIP lowered the NSB SC tissue matrix interaction of mAb 1RE relative to mAb 1P which improved the in vivo PK of mAb 1RE. Under the conditions leveraged in this study, the temperatures at which the onset thermal instability and aggregation occurred for the two mAbs was marginally dissimilar with mAb 1RE (re-engineered molecule) showing slightly lower values in both aspects than mAb 1P. The data suggest for molecules with little/no hydrophobic interaction potential, but high local charge-related physiochemical aspects, thermal instability and aggregation are not distinguishing factors. The improved SC absorption and bioavailability for mAb 1RE are likely related to reduced non-specific tissue uptake and subsequent catabolism compared to mAb 1P, which is supported by earlier studies showing enhanced NSB driven cellular association/binding led to mAb degradation (Datta-Mannan A, et al., mAbs 2015; 7:483-93). Interestingly, while the relative rank order of the SC PK of mAb 1RE and mAb 1P were the same in cynomolgus monkeys and rats, differences in the magnitude of ka and bioavailability were observed across species. This may be related to the known differences in the compositional preponderance of the components within SC tissue matrix across species; these SC tissue compositional differences across species may also be impacting the correlation analyses across species. Nonetheless, while the magnitude of the effects may not be fully predictable across species because of the differences in SC tissue architecture, the non-specific nature of the charge-based interactions strongly suggests similar findings would be anticipated in humans.

In contrast to the Platform 1, Platforms 2 and 3 facilitated dissection/connectivity of the impact varying levels of both charge-related NSB and hydrophobic-related interactions have on SC absorption and bioavailability. The Platform 2 mAb pair served as a reasonable set of molecules to dissect the role of charge-based interactions (mAb 2P) shows ˜2-fold higher HpnTP than mAb 2RE with an underlying similar hydrophobic interaction component (mAbs 2P and 2RE have HIP values of ˜16% and ˜20%, respectfully). The charge re-engineering improved the kinetics (i.e. clearance) and SC absorption/bioavailability of mAb 2RE compared to mAb 2P by ˜4.5-fold and ˜3-fold, respectively, in rats. Thus, similar to Platform 1 findings, the Platform 2 data also supports positive charge-based interactions negatively affect SC absorption/bioavailability likely through increased binding/association with the components of SC tissue matrix. It is, however, important to note that although charge rebalancing significantly improved the PK, mAb 2RE still displays a relative clearance rate and SC bioavailability of ˜1 mL/hr/kg and ˜60%, respectively, in rats. The engineered mAb 2RE is actually kinetically inferior to the charge unbalanced parental mAb 1P (CL/F and SC % F of ˜0.8 mL/hg/kg and ˜70%, respectively) even though mAbs 1P and mAb 2RE have similar HpnIP values. The major differences between the mAb 1P and the mAb 2RE variants in their HIP values, which show mAb 2RE has an ˜20-fold higher hydrophobic interaction potential than mAb 1P. Taken together, the data suggest that the hydrophobic interaction potentials for the Platform 2 mAbs are an important component of NSB influencing both mAbs 2P and 2RE clearance, SC absorption and bioavailability. The data suggest further engineering to reduce the HIP of mAbs 2P and 2RE would likely improve their SC absorption through reduced interactions with the SC tissue matrix.

Similar to Platform 2, the Platform 3 mAbs were also insightful for dissecting the role of hydrophobic interactions on the rate and extent of mAb SC absorption and bioavailability. Platform 3 is unique from Platform 2 in that the mAbs in Platform 3 show larger differences in their hydrophobic interactions (˜6-9-fold HIP differences for Platform 3 mAbs whereas Platform 2 mAbs have comparable HIP values) and some charge based interactions in a more moderate range (HpnTP values of ˜20-46% for mAbs 3P, 3RE1 and 3RE2 compared to >58% for mAbs 2P and mAb 2RE). Thus, while not fully seamless with regard to HIP and HpnIP, Platform 3 does directionally facilitate the interrogation of hydrophobicity with a more modest influence from charge than the other two Platforms. The ˜9-fold higher HIP value for mAb 3P compared to mAb 3RE1 was connected to an ˜1.6-fold more rapid clearance and ˜2-fold lower bioavailability of mAb 3P than mAb 3RE1 in cynomolgus monkeys, respectfully, and an ˜2.3-fold more rapid clearance and ˜1.7-fold lower bioavailability of mAb 3P compared to mAb 3RE1 in rats, respectfully. The ˜6-fold higher HIP value for mAb 3P compared to mAb 3RE2 was connected to an ˜2-fold more rapid clearance and ˜comparable bioavailability of mAb 3P compared to mAb 3RE2 in cynomolgus monkeys, respectfully, and an ˜2.2-fold more rapid clearance and ˜2-fold lower bioavailability of mAb 3P compared to mAb 3RE2 in rats, respectfully. Thus, it is apparent from Platform 3 reducing the hydrophobic interactions was of benefit to the PK; however, when compared to the PK enhancements observed for Platform 2 (˜4.5-fold and ˜3-fold improvements in clearance and SC bioavailability, respectively) the Platform 3 improvements were more modest. The findings suggest that for the molecules herein, when both charge and hydrophobicity interactions are present, reducing charge-based interactions may have a greater impact on enhancing kinetics than reducing hydrophobic interactions. Notably, the high correlation across Platforms between the slow clearance (or CL/F for SC route) and reduced HIP, as well as, decreased HpnIP for both the IV and SC administration does indicate reducing hydrophobic interactions or charge based association will improve PK for both parenteral routes across species; thus, it is important to optimize molecules for both these properties. Furthermore dissection, of the Cₘₐₓ across Platforms suggests SC route specific high correlations with T_{agg} and T_{m onset}. This may be related to thermal stability playing an increased role within the SC tissue matrix prior to absorption into the blood circulation. A high correlation was generally not consistently observed across rats and cynomolgus monkeys between ka or SC bioavailability and the majority of the physiochemical properties (with the exception of T_{m onset} and kₐ), across Platforms. While this may in part be due to the number of mAbs examined, the reasonable connectivity of some of these parameters in spite of the limited examples, indicates further study with additional molecules is warranted to better delineate these relationships both within and across species.

The findings suggest mAb variants that have an increased rate of SC absorption and bioavailability of mAbs have reduced local positive charge potentially, lower hydrophobic matrix interactions, higher thermal stability and reduced thermally induced aggregation potential. These observations lend to hypothesizing that the results may be related to a few mechanisms: 1) decreased SC tissue matrix interactions due to charge repulsion with the negatively charged components of the SC milieu including constituents of the ECM including GAGs; 2) reduced SC tissue component interactions due to inferior hydrophobic based van der Waals interactions with adipose tissues; and 3) reduced potential for local and global unfolding due to physiologically relevant temperatures which may lead to increased recognition by macrophage based host defense mechanisms in the SC space. The reduced clearance of mAbs 1RE, 2RE and 3RE1 and 3RE2 (relative to mAbs 1P, 2P and 3P, respectively) is likely a consequence of these mechanisms, but to variable degrees. In particular, the reduced SC absorption and bioavailability of mAbs 1P and 3P correlate well with the high degree of local SC tissue association and subsequent degradation due to a combination of increased charge- or hydrophobic-based interactions, respectively. Although tissue binding data was not assessed for the Platform 2 molecules due to TMDD in cynomolgus monkeys, for mAb 2P, the preponderance of data suggests a likely increased degree of local SC tissue binding due to both charge and hydrophobic interactions. From a conceptual perspective, SC administered kinetically poorer mAbs likely bind GAGs, fat lobules and cells (adipocytes and endothelia) non-specifically to a greater extent than molecules without these properties. As a result, the greater degree/strength of association with SC tissue components, does not allow the mAbs to be taken into the lymphatic system for subsequent release into the peripheral circulation. In addition, increasing endothelial cellular association with membrane components may lead to the mAb's increased cellular uptake but lack of ability to be effectively salvaged from intracellular degradation. Due to the non-specific nature of the interactions, this may partition the mAbs with solvent exposed charge and increased hydrophobic potential properties away from the recycling pathway and towards lysosomal degradation. Lastly, local tissue interactions and sensitivity to the higher physiological temperatures in vivo maybe facilitating some loss of tertiary structure for some molecules which leads to some increased aggregation potential. These aggregates may appear ‘foreign’ to the system and facilitate an increased response by SC macrophages to facilitate removal (i.e. degradation) of the mAbs, so that these are no longer available for absorption. This seems to make sense if one considers both the preponderance physiochemical findings in the context of the potential cellular and SC tissue matrix interactions as discussed above.

In summary, the data shown here suggest there are many mAb-based and SC matrix centric factors to consider when utilizing antibody engineering or screening approaches to improve the SC bioavailability/drug-ability of mAb biotherapeutics. Applying a rationally-based approach to integrate the complexities of these factors impacts the in vivo performance of mAbs. Since additional characteristics of both the mAb and formulation (chemical stability, FcRn binding, solubility, concentrate-ability) can influence the SC disposition and elimination it will be impactful to investigate the relative roles of these additional mechanisms to ultimately design, engineer and screen molecules with increased therapeutic potential.

SEQUENCE LISTING
SEQ ID NO: 1
KSSQSLLYSRGKTYLN
SEQ ID NO: 2
AVSKLDS
SEQ ID NO: 3
VQGTHYPFT
SEQ ID NO: 4
GYTFTRYYIN
SEQ ID NO: 5
INPGSGNTKYNEKFKG
SEQ ID NO: 6
EGTTVY
SEQ ID NO: 7
GYTFTDYYIN
SEQ ID NO: 8
EGETVY
SEQ ID NO: 9
RASKSISKYTA
SEQ ID NO: 10
AGSKRHW
SEQ ID NO: 11
QQHNEYPYT
SEQ ID NO: 12
GYAFTSFLIE
SEQ ID NO: 13
SNPRTGRTKYKSKFRG
SEQ ID NO: 14
EFFDY
SEQ ID NO: 15
AGSKLHW
SEQ ID NO: 16
SNPRTGGRKYKEKFRG
SEQ ID NO: 17
RSSQSLLISGGKTYLN
SEQ ID NO: 18
LVSKLDQ
SEQ ID NO: 19
WQGTYFPLT
SEQ ID NO: 20
GKTFWSYGIN
SEQ ID NO: 21
IYIGTGYTEPNPKYKG
SEQ ID NO: 22
IGGYYGNFDQ
SEQ ID NO: 23
KASDHIGKFLT
SEQ ID NO: 24
GATSKLT
SEQ ID NO: 25
QQYWSTPFT
SEQ ID NO: 26
GYKFTRYVMH
SEQ ID NO: 27
INPYNDGVNYNEKFKG
SEQ ID NO: 28
NWDTGL
SEQ ID NO: 29
KASDHILKFLT
SEQ ID NO: 30
GATSLET
SEQ ID NO: 31
QMYWSTPFT
SEQ ID NO: 32
GYKFTRYVMH
SEQ ID NO: 33
INPYNDGTNYNEKFKG

1. A method of selecting an antibody suitable for subcutaneous administration, the method comprising:
measuring T_{agg} (temperature of aggregation onset) of a first and a second antibody that binds to the same target,
measuring T_{m onset} (temperature of the unfolding onset) of the first and second antibody, comparing the T_{agg} and T_{m onset} of the first and second antibody; and
selecting the first or second antibody that has a higher T_{agg} and/or T_{m onset} for subcutaneous administration.

2. The method of claim 1, wherein the method further comprises measuring HpnIP (heparin binding interaction potential) and/or HIP (hydrophobic interaction potential) of the first and second antibody.

3. The method of claim 1, wherein the method further comprises measuring the rate of subcutaneous absorption (ka) and/or subcutaneous bioavailability (% F) of the first and second antibody.

4. The method of claim 1, wherein the method further comprises measuring one or more of the pharmacokinetics (PK) parameters of the first and second antibody, wherein the PK parameters are selected from Cₘₐₓ (maximal observed serum concentration), Tₘₐₓ (time of maximal observed serum concentration), AUC_{0-inf} (area under the serum concentration curve from time zero extrapolated to infinite time), CL/F (clearance following SC administration), and T_{1/2} (elimination half-life).

5. The method of claim 1, wherein the method further comprises selecting the first or second antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid;
the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

6. The method of claim 1, wherein the method further comprises selecting the first or second antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

7. (canceled)

8. A method of generating a variant antibody with improved subcutaneous absorption and bioavailability compared to a parental antibody, the method comprising:
generating a variant antibody of the parental antibody, wherein the variant antibody has a higher T_{agg} and/or T_{m onset} than the parental antibody, and wherein the variant antibody has a lower HpnIP and/or HIP than the parental antibody.

9. (canceled)

10. The method of claim 8, wherein the method further comprises generating a variant antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid;
the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

11. The method of claim 8, wherein the method further comprises:
replacing the amino acid residue at position 24 of LCDR1 of the parental antibody with lysine;
replacing the amino acid residue at position 54 of LCDR2 of the parental antibody with leucine;
replacing the amino acid residue at position 55 of LCDR2 of the parental antibody with aspartic acid or glutamic acid;
replacing the amino acid residue at position 56 of LCDR2 of the parental antibody with serine or threonine;
replacing the amino acid residue at position 96 of LCDR3 of the parental antibody with phenylalanine; or
replacing the amino acid residue at position 61 of HCDR2 of the parental antibody with glutamic acid;
wherein all positions are numbered according to Kabat numbering and the CDRs are defined by a hybrid of Kabat and Chothia.

12. The method of claim 8, wherein the method further comprises generating a variant antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

13. The method of claim 8, wherein the method further comprises:
replacing the amino acid residue at position 25 of LCDR1 of the parental antibody with alanine or serine;
replacing the amino acid residue at position 26 of LCDR1 of the parental antibody with serine;
replacing the amino acid residue at position 52 of LCDR2 of the parental antibody with serine or threonine;
replacing the amino acid residue at position 89 of LCDR3 of the parental antibody with glutamine or valine;
replacing the amino acid residue at position 90 of LCDR3 of the parental antibody with glutamine;
replacing the amino acid residue at position 95 of LCDR3 of the parental antibody with proline;
replacing the amino acid residue at position 97 of LCDR3 of the parental antibody with threonine;
replacing the amino acid residue at position 26 of HCDR1 of the parental antibody with glycine;
replacing the amino acid residue at position 27 of HCDR1 of the parental antibody with tyrosine;
replacing the amino acid residue at position 29 of HCDR1 of the parental antibody with phenylalanine;
replacing the amino acid residue at position 30 of HCDR1 of the parental antibody with threonine;
replacing the amino acid residue at position 62 of HCDR2 of the parental antibody with lysine; or
replacing the amino acid residue at position 65 of HCDR2 of the parental antibody with glycine;
wherein all positions are numbered according to Kabat numbering and the CDRs are defined by a hybrid of Kabat and Chothia.

14. The method of claim 8, wherein the method further comprises measuring ka and/or % F of the parental antibody and the variant antibody.

15. The method of claim 8, wherein the method further comprises measuring one or more of the PK parameters of the parental antibody and the variant antibody, wherein the PK parameters are selected from Cₘₐₓ, Tₘₐₓ, AUC_{0-inf}, CL/F, and T_{1/2}.

16. (canceled)

17. A method of selecting an antibody suitable for subcutaneous administration, the method comprising selecting an antibody that comprises one or more of the following amino acid residues:
the amino acid residue at position 24 of LCDR1 is lysine;
the amino acid residue at position 54 of LCDR2 is leucine;
the amino acid residue at position 55 of LCDR2 is aspartic acid or glutamic acid;
the amino acid residue at position 56 of LCDR2 is serine or threonine;
the amino acid residue at position 96 of LCDR3 is phenylalanine; or
the amino acid residue at position 61 of HCDR2 is glutamic acid;
wherein all positions are numbered according to Kabat numbering and the CDRs are defined by a hybrid of Kabat and Chothia.

18. The method of claim 17, wherein the method further comprises selecting an antibody that comprises one or more of the following amino acid residues:
the amino acid residue at position 25 of LCDR1 is alanine or serine;
the amino acid residue at position 26 of LCDR1 is serine;
the amino acid residue at position 52 of LCDR2 is serine or threonine;
the amino acid residue at position 89 of LCDR3 is glutamine or valine;
the amino acid residue at position 90 of LCDR3 is glutamine;
the amino acid residue at position 95 of LCDR3 is proline;
the amino acid residue at position 97 of LCDR3 is threonine;
the amino acid residue at position 26 of HCDR1 is glycine;
the amino acid residue at position 27 of HCDR1 is tyrosine;
the amino acid residue at position 29 of HCDR1 is phenylalanine;
the amino acid residue at position 30 of HCDR1 is threonine;
the amino acid residue at position 62 of HCDR2 is lysine; or
the amino acid residue at position 65 of HCDR2 is glycine;
wherein all positions are numbered according to Kabat numbering and the CDRs are defined by a hybrid of Kabat and Chothia.

19. The method of claim 17, wherein the method further comprises measuring T_{agg} and T_{m onset} of the antibody.

20. The method of claim 17, wherein the method further comprises measuring HpnIP and/or HIP of the antibody.

21. The method of claim 17, wherein the method further comprises measuring ka and/or % F of the antibody.

22. The method of claim 17, wherein the method further comprises measuring one or more of the PK parameters of the antibody, wherein the PK parameters are selected from Cₘₐₓ, Tₘₐₓ, AUC_{0-inf}, CL/F, and T_{1/2}.

23. (canceled)

24. A method of administering an antibody to a subject subcutaneously, the method comprising:
measuring T_{agg} and T_{m onset} of the antibody,
determining the antibody is suitable for subcutaneous administration, and
subcutaneously administering the antibody to the subject.

25. The method of claim 24, the method further comprises measuring HpnIP and/or HIP of the antibody.

26. The method of claim 24, the method further comprises measuring ka and/or % F of the antibody.

27. The method of claim 24, the method further comprises measuring one or more of the PK parameters of the antibody, wherein the PK parameters are selected from Cₘₐₓ, Tₘₐₓ, AUC_{0-inf}, CL/F, and T_{1/2}.

28. The method of claim 24, wherein the antibody is a monoclonal antibody.

29. The method of claim 28, wherein the monoclonal antibody has an IgG1 or IgG4 isotype.

30.-31. (canceled)

32. The method of claim 1, wherein the antibody is a monoclonal antibody.

33. The method of claim 32, wherein the monoclonal antibody has an IgG1 or IgG4 isotype.

34. The method of claim 8, wherein the antibody is a monoclonal antibody.

35. The method of claim 34, wherein the monoclonal antibody has an IgG1 or IgG4 isotype.

36. The method of claim 17, wherein the antibody is a monoclonal antibody.

37. The method of claim 36, wherein the monoclonal antibody has an IgG1 or IgG4 isotype.
